(19) United States
(12) Patent Application Publication
(10) Pub. No.: US 2006/0165811 A1
(43) Pub. Date: Jul. 27, 2006

(54) Method and system for automatic decoding of motor cortical activity

(76) Inventors: Michael J. Black, Providence, RI (US); Frank Wood, Providence, RI (US); Wei Wu, Chicago, IL (US)

Correspondence Address: Joseph P. Quinn, Esq.;Brown Rudnick Berlack Israels LLP, One Financial Center, Boston, MA, 02111, US

(21) Appl. No.: 11/086,956

(22) Filed: Mar. 22, 2005

Related U.S. Application Data
(60) Provisional application No. 60/647,889, filed on Jan. 26, 2005.

(51) Int. Cl. A61K 35/30

(52) U.S. Cl. 424/570

(57) ABSTRACT

A Switching Kalman Filter Model for the real-time inference of hand kinematics from a population of motor cortical neurons. Firing rates are modeled as a Gaussian mixture where the mean of each Gaussian component is a linear function of hand kinematics. A “hidden state” models the probability of each mixture component and evolves over time in a Markov chain. Gaussian mixture models and Expectation Maximization (EM) techniques are extended for automatic spike sorting. Good initialization of EM is achieved via spectral clustering. To account for noise, the mixture model is extended to include a uniform outlier process. A greedy optimization algorithm that selects models with different numbers of neurons according to their decoding accuracy is used to automatically determine the number of neurons recorded per electrode. Closed loop neural control of external events are demonstrated using neural control of a computer curser.

### RELATED APPLICATION

The present application claims the benefit of U.S. provisional application 60/647,889 filed on Jan. 26, 2005, which is incorporated herein by reference.

### FIELD OF THE INVENTION

The present invention relates to the field of biomedical engineering and more particularly to the field of interpreting neural signals.

### BACKGROUND OF THE INVENTION

Recent research has demonstrated the feasibility of continuous neural control of devices such as computer cursors using implanted electrodes. These results are enabled by a variety of mathematical “decoding” methods that produce an estimate of the system “state” (e.g. hand position) from a sequence of measurements (e.g. the firing rates of a population of cells).

Current research focuses on the real-time decoding of a continuous movement signal from population activity in the arm area of primary motor cortex (MI). The primary methods for decoding MI activity include the population vector algorithm, linear filtering, artificial neural networks, and probabilistic methods. The majority of these approaches model a linear relationship between the firing rates of motor cortical neurons and hand kinematics.

The population vector approach is the oldest method and was pioneered by Georgopoulos and colleagues in early 1980's to model the encoding of hand movement direction by a population of MI neurons. (A. Georgopoulos, et al “On the relations between the direction of two-dimensional arm movement and cell discharge in primate motor cortex” Journal of Neuroscience, vol. 8, no. 11, pp. 1527-1537, 1982; A. Georgopolos, et al. “Neural population coding of movement direction” Science, vol. 233, pp. 1416-1419, 1986.) This work has led to a number of further observations that show that motor cortical firing rates are related to hand position, velocity (movement direction and speed) and acceleration. The population vector algorithm has been used successfully to decode various hand movement tasks which include center-out reaching, sinusoid tracing, spiral tracing and lemniscate tracing. Recently, the population vector algorithm was applied to the real-time neural control of 3D cursor movement in a center-out reaching task (D. Taylor et al., “Direct cortical control of 3D neuroprosthetic devices, “Science, vol. 296, no. 5547, pp. 1829-1832, 2002).

Although the population vector approach has shown success in these decoding and neural control tasks, it makes a number of assumptions which are difficult to achieve in practical situations. For example, it assumes that the population uniformly represents the space of movement directions. With small populations this is often not satisfied. Additionally the population vector method implicitly assumes a linear relationship between hand velocity and firing rate. This has been shown many times to be a reasonable approximation when the data comes from well isolated cells. If activity from multiple units is combined by poor on-line spike sorting, this assumption may be violated.

The population vector method typically focuses on the relationship between hand velocity and firing rates. Continuous decoding of hand position then requires integration of velocity estimates over time. For general motions, errors quickly propagate reducing the effectiveness of the method. Consequently, the approach is most often used for tasks with stereotyped motions such as reaching or for on-line control where the subject may adapt to compensate for deficiencies in the decoding method.

An alternative model uses linear regression to compute fixed linear filters relating hand position to a vector of firing rates defined over a relatively long time window (typically 500 ms to 1.5 s). Due to its accuracy and efficiency, this linear regression model has been successfully applied to real-time direct neural control tasks.

Artificial neural networks have also been applied to neural decoding problems and shown to be successful in the real-time prediction of hand trajectory from neural ensembles in MI. The results were not significantly different from that of the linear filter and, recently, an analysis of the representation learned by such networks reveals that they essentially encode a linear model (cosine tuning) as in the population vector method.

The artificial neural networks, as well as population vectors and linear filters, lack both a clear probabilistic model and a model of the temporal hand kinematics. Additionally, they provide no estimate of uncertainty and hence may be difficult to extend to the analysis of more complex temporal movement patterns. In contrast, a probabilistic formulation allows the mathematically principled combination of information and can take into account uncertainty in the data.

The benefits of a probabilistic approach that uses data in small time windows (e.g. 50-100 ms or less) and integrates that information over time in a recursive fashion, has previously been suggested. In particular; the present inventors recently proposed a Kalman filter, in which hand movement is encoded by a population of cells with a linear Gaussian model and is decoded using the Kalman filter algorithm. The method is based on an approximate generative model of neural firing. In particular, it assumes that the observed firing rates are a linear function of hand kinematics (position, velocity, and acceleration) and that they are corrupted by Gaussian noise.

This generative model is only a rough approximation and is violated in many ways. The distribution of firing rates is not Gaussian and the relationship to hand kinematics is only approximately linear. Moreover, poor spike sorting can compound these problems resulting in decreased decoding performance for any linear approach. This can occur, for example, when a single electrode records the activity of multiple cells with similar waveforms that are difficult to distinguish.

Traditional neuroscience involves the study of small populations of cells where neural activity is recorded and manually processed for ‘off-line’ analysis. However, neural prosthesis applications involve simultaneously recording from hundreds of electrodes and decoding neural activity ‘on-line’. These constraints make hand sorting of neural data impractical for prosthesis applications and highlight the need for automated methods.

Traditionally, the goal of spike sorting has been to identify the activity of individual cells for later analysis. However, neural prostheses require near instantaneous decoding. Accordingly, an automated spike sorting algorithm is needed that optimizes decoding accuracy for neural control tasks.

Efforts to improve decoding have led to the adoption of several powerful algorithms such as Kalman filtering and particle filtering. These heretofore known methods have yielded improvements over earlier population coding and linear filtering approaches. All of these decoding algorithms posit and exploit some function relating the firing rate of a population of cells to kinematic data such as hand position. However, it is difficult to determine the firing rate of a population of cells accurately.

Firing rates can be determined by counting the number of spikes a neuron produces in some time window (or bin), but detecting and assigning spikes to neurons is difficult to do. This is especially true when recording with the chronically implantable, relatively low-impedance multi-electrode arrays desirable for use in neuroprosthetic applications. Experts often disagree about what is and is not a spike and about the number of neurons present in recordings for a given electrode. Similar variability has been found between experts.

A number of spike sorting techniques have previously been investigated. These techniques include template matching and k-means or Gaussian mixture model clustering of waveform principle components. Neural networks and t-distribution mixture model clustering techniques have also been developed.

None of the methods above adequately addressed either automatically determining the number of units recorded or identifying noise. Certain heretofore known methods appear to identify a high number of units for real data. Likelihood penalties have been used in other methods to select the correct number of components in a Gaussian mixture model. It has been found that likelihood penalties such as the Bayesian and Aikaike information criteria tend to select models with too many component mixtures.

Sophisticated algorithms are heretofore known for detecting and sorting spikes from tetrodes. These algorithms exploit dependencies between recorded waveforms arising from tetrodes' small intra-recording-tip distances. The intra-tip distance of multi-electrode arrays are large enough that these dependencies cannot be exploited. It is easier to detect spikes in single-cell intra- and extra-cellular recordings because of high signal to noise ratios due to controlled placement of the electrode tip. However, such controlled placement appears impractical for chronic neuro-prosthetic applications.

### SUMMARY OF THE INVENTION

The present invention provides a Switching Kalman Filter Model for the real-time inference of hand kinematics from a population of motor cortical neurons. Firing rates are modeled as a Gaussian mixture where the mean of each Gaussian component is a linear function of hand kinematics. A “hidden state” models the probability of each mixture component and evolves over time in a Markov chain. The model generalizes previous encoding and decoding methods, addresses the non-Gaussian nature of firing rates, and can cope with crudely sorted neural data common in on-line prosthetic applications.

The new model according to the present invention is based on the Switching Kalman Filter. This model generalizes previous approaches, achieves real-time decoding, and is appropriate for continuous neural-prosthetic control tasks.

The probability of the firing rates of the population is modeled at an instant in time as a Gaussian mixture where the mean of each Gaussian is a linear function of the hand kinematics. This mixture contains a “hidden state,” or weight, that assigns a probability to each linear, Gaussian, term in the mixture. The evolution of this hidden state over time is modeled as a Markov chain. The Expectation-Maximization (EM) algorithm is used to fit this mixture model to training data that consists of measured hand kinematics (position, velocity, acceleration) and the firing rates of a small population of cells recorded with a chronically implanted multi-electrode array. Decoding of neural test data is achieved using the Switching Kalman Filter (SKF) algorithm. Quantitative results show that the SKF outperforms the traditional linear Gaussian Kalman filter in the decoding of hand movement. These results suggest that the SKF provides a real-time decoding algorithm that may be appropriate for neural prosthesis applications.

The approach addresses a number of key issues and by doing so improves decoding accuracy over previous methods. First, the mixture model represents non-Gaussian distributions of firing rates. Previously, particle filtering has been proposed for modeling and de-coding arbitrary, non-Gaussian, neural activity. While general, this approach is computationally expensive and currently inappropriate for real-time decoding. The SKF can model non-Gaussian activity while maintaining many of the computational advantages of traditional linear Gaussian models; this is critical for neural prostheses. The SKF approach also addresses a common problem with on-line neural data. In prosthetic applications, individual electrodes may pick up activity of multiple cells and on-line spike detection and sorting techniques must be employed These techniques tend to be based on simple thresholds and waveform analysis and may result in multiple units being classified as a single cell. In this respect, prosthetic applications differ somewhat from work on off-line encoding/decoding where careful spike sorting may be possible.

It is desirable to systematically extend the Kalman filter encoding model to non-linear relationships and non-Gaussian statistics and to evaluate the performance of such a model with respect to neural decoding. Unfortunately, fully general non-linear models are difficult to train from training data and the associated decoding methods are computationally expensive.

Instead, the SKF model described herein treats neural firing rates as a probabilistic mixture of linear models. If the encoding model of each cell is linear, then the SKF is better able to approximate the combination of firing rates. The method extends the Kalman model to exploit a mixture of linear Gaussian models. This mixture model, combined with a linear Gaussian model for the hand kinematics, is referred to as Switching Kalman Filter Model (SKFM). It is also known as hybrid model, state-space model with switching, jump, linear system and conditional dynamic linear model.

While such a model is more general than the simple linear Gaussian model, it still admits an efficient, real-time, decoding algorithm. Generalization to non-Gaussian firing models and methods to cope with poor spike sorting are shown. In particular, test data is constructed that is intentionally corrupted to simulate the effect of poor spike sorting. The firing rates of pairs of well isolated units are summed to produce synthetic mixed cells and it is demonstrated that the SKFM is able to separate the combined activity to approximate the linear tuning properties of the individual units. The method is also tested on data recorded during a neural prosthetic control task. While on-line control is not tested here, the off-line reconstruction results demonstrate the appropriateness of the method for decoding and suggest that on-line experiments should be pursued.

The present invention also provides an automatic spike sorting method and system that produces decoding results as good or better than those obtained by the best human spike sorter. Kalman filtering is used to decode hand trajectories from neural recordings made in the motor cortex of a monkey performing a cursor control task. Gaussian mixture models and Expectation Maximization (EM) techniques are extended for automatic spike sorting. Good initialization of EM is achieved via spectral clustering. To account for noise, the mixture model is extended to include a uniform outlier process. A greedy optimization algorithm that selects models with different numbers of neurons according to their decoding accuracy is used to automatically determine the number of neurons recorded per electrode. Data recorded from motor cortex is used evaluate performance with respect to the decoding of hand position from firing rates. Spike trains obtained by the present automated technique provide more accurate neural decoding than spike trains obtained by human expert sorting.

Although there are many ways to record neural activity, the present invention focuses on spike sorting data recorded from multi-electrode arrays such as the Bionic Intracortical Array. Spike sorting includes determining which waveforms were noise, how many neurons ere recorded, and which neuron each non-noise waveform came from. Methods of spike detection are not addressed herein because it is assumed that waveforms sorted were captured after crossing experimentally determined thresholds.

The present invention is an extension of Gaussian mixture model clustering. Although it has been suggested that t-distributions better represent the distribution of waveforms generated by a single neuron, methods according to the present invention have produced satisfactory results using mixtures of Gaussians, and a uniform noise process.

The present invention includes an automatic spike sorting process that is an extension of the Gaussian mixture model Expectation Maximization (EM) technique reviewed by M. S. Lewicki, “A review of methods for spike sorting: the detection and classification of neural action potentials,” Network, vol. 9, no. 4, pp. R53-78, 1998. In an illustrative embodiment, the present inventive process initializes the mixture model distribution parameters by clustering a subset of the data by spectral clustering. Model selection is performed by greedily optimizing decoding error.

Illustrative embodiments of the inventive process decode using models with varying numbers of densities for each channel of a recording. The model that produces the best decoding results is kept. Decoding accuracy is used as an objective means of evaluating spike sorting performance. Such evaluation has determined that the method of the present invention performs as well as or better than expert human sorters.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 is a graphical model representation for an exemplary Switching Kalman Filter Model;

FIG. 2 is a graphical output of four test trial using a Switching Kalman Filter Model to reconstruct true hand trajectory;

FIG. 3 is a graphical distribution of empirical firing rates compared to fitted models;

FIG. 4 is a graphical representation of each component of the system state motion for true and reconstructed motion using a Switching Kalman Filter;

FIG. 5 is a graphical representation of confidence estimation for x and y position comparing true and reconstructed trajectories.

FIG. 6 is a greedy sorter algorithm;

FIG. 7 is graphical representation of reconstructed x and y positions using a Kalman filter method;

FIG. 8 is a graphical representation of reconstructed x and y position data using a linear regression method;

FIG. 9 is a histogram of time-to-target using a Kalman filter decoding method;

FIG. 10 is a graphical representation of x position data using a linear regression method with smoothing;

FIG. 11 is graphical representation of the logarithm of power spectra of true x position and reconstructed x position using a Kalman filter method; and

FIG. 12 is a graphical representation of logarithm of power spectra of true x-position and reconstructed x-position using a Kalman filter with smoothed firing rates.

### DETAILED DESCRIPTION

Data Acquisition and Processing

To describe the SKF model and its application to neural decoding, two datasets consisting of simultaneously recorded hand kinematics and neural activity are considered. Both experiments used neural signals recorded with Bionic Technologies LLC (BTL) 100-electrode silicon arrays which were chronically implanted in the arm area of primary motor cortex (MI) in macaque monkeys. Signals were amplified and sampled at 40 kHz/channel using a commercial recording system. The experiments differ in the task being performed and the processing of the recorded waveforms.

The specific design of the task will effect the resulting encoding model. The common radial reaching tasks vary the direction of movement and consequently encoding models using this data focus on directional tuning. More general control tasks (e.g. computer cursor control) require full two-dimensional control (at least). Consequently, two tasks in which the hand motion spans a range of 2D positions, velocities, and accelerations are considered.

First, in a “Pursuit tracking task”, Electrophysiological recordings were made while the monkey performed a continuous tracking task. The monkey viewed a computer monitor while gripping a two-link manipulandum that controlled the 2D motion of a cursor on the monitor. In each trial, a target dot moved slowly and continuously on the monitor and the task required moving a feedback dot with the manipulandum so that it kept tracking the target within a given distance range. Hand positions were recorded every 50 ms and from this velocity and acceleration were computed using simple differencing.

A trial ended when the dot fell outside the tracking range. The short trials (with duration less than 5 seconds) were eliminated. The majority of the remaining 182 trials were approximately 8-9 seconds in duration. Note that the hand motions in this task were more general than those in the more common stereotyped tasks such as “center-out” tasks, in that the motions spanned a range of directions, positions, and speeds. They cannot, however, be considered natural motions.

During a trial, all neural activity that crossed a manually set threshold was digitized (12-bit voltage resolution) and saved to disk. Waveforms and their corresponding timestamps were saved for each electrode in the array. These waveforms were “sorted” by hand (off-line) using a commercial software. Twenty five well isolated units were detected and used for analysis. The empirical firing rate for each unit was calculated by counting the number of spikes within the previous 50 ms time window. The mean firing rate for each unit was then subtracted to obtain zero-mean data

Second, in a “Pinball” task, a target dot appeared at a random location on the monitor and the monkey was required to move the feedback dot with the manipulandum to hit the target When the target was hit, it randomly jumped to a new 2D location. In this task, the subject's hand motions were fast and unconstrained; this was more natural than the motion in the pursuit tracking task and simulated the motions needed to control a computer interface.

Each trial in this task was typically several minutes long. The data analyzed here includes two trials: one of approximately 3.5 minutes in length was used as training data and the other, approximately 1 minute long, was used as test data for decoding.

As in the off-line task, hand position was recorded and hand velocity and acceleration were computed from the hand positions. Here, however, the task was designed for on-line neural control and, consequently, the spike acquisition exploited simple thresholding of waveforms rather than manual, off-line, sorting. As a result, the activity recorded with a single electrode may be the combination of the spiking activity of multiple cells. Data was recorded from 42 channels, action potentials crossing manually set thresholds were detected, and the firing rate for each channel was computed in 70 ms time bins. The mean firing rates in this task were larger than that in the pursuit tracking task due to the more rapid motions and the possible combination of units. The distribution of firing rates was also less Gaussian so a square-root transform was applied to the firing data. The mean firing rate for each unit was then subtracted to obtain zero-mean data.

Data Pre-Processing

In following description, a Gaussian mixture model is fitted to the firing data in which each component of the model had a full covariance matrix (i.e. 42×42). Given the large number of units, correlations between their firing activity, and a limited amount of training data, fitting multiple covariance matrices can be computationally infeasible. To deal with this issue, the dimensionality of the input firing rates is reduced using Principal Component Analysis (PCA).

Let zₜ=[zᵢ,ₜ, . . . , z, . . . ,t]^{T}εRⁿ represent a the zero-mean firing rates of n cells at time bin t. A matrix A=[z₁ . . . z^{m}] was constructed for the M time bins in the training data. Since the mean firing rate for each cell is zero, the covariance matrix Σ of the firing rate vectors is given by Σ=AA^{T}. Since Σ is symmetric and positive semi-definite, there exists an orthonormal matrix U and a non-negative diagonal matrix Λ such that Σ=UΛU^{T}. Each column in U is an eigerrvector and each diagonal entry in Λ is an eigenvalue which reveals the variance in the corresponding eigenvector direction. The total variance is the sum of all these eigenvalues. If the firing rates of cells covary then the covariance matrix will not be full rank and some of the eigenvalues will be small. In this case fitting the covariance matrices in the next section will be ill-conditioned.

To cope with this problem, let U be a sub-matrix of U which only contains those eigenvectors with correspondingly large eigenvalues. Let Ź=U^{T}Λ, be the projection of the original firing rate vectors on the lower dimensional subspace spanned by the columns of U. In the pinball task data, the projection of the 42 firing rates onto a 39 dimensional subspace resulted in a loss of less than 1% of the total variance. Rather than work with the original firing rate, their projections were used wherein and each column in Ź was referred to as a vector of firing rates for simplicity.

Computational Methods

In the Switching Kalman Filter Model, the hand movement (position, velocity and acceleration) is modeled as the system state and the neural firing rate is modeled as the observation (measurement). Let the state of the hand at the current instant in time be Xₜ=[x, y, vₓ, v_{y}, aₓ, a_{y}]T₁εR⁶a, which represents x-position, y-position, x-velocity, y-veloci and y-acceleration at time tΔt where Δt=70 ms (pinball task) or 50 ms (off-line task) in the experiments. The observations yₜεR^{K} represent a K×1 vector containing the firing rates at time t for the K observed neurons within Δt.

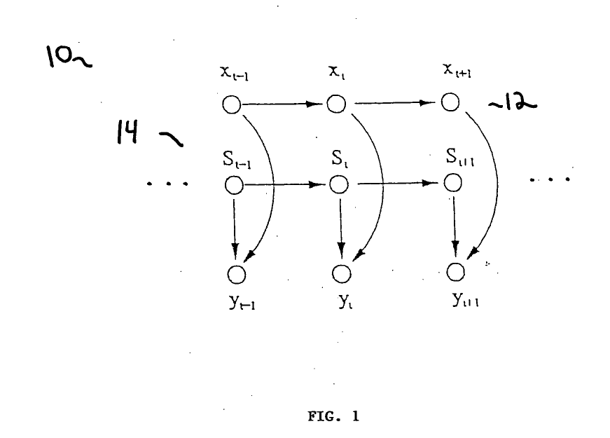

It is desirable to model the probabilistic relationship between Xₜ and Yₜ. To that end, the switching Kalman filter (SKF) model illustrated in FIG. 1 is used. FIG. 1 is a graphical model representation for the switching Kalman filter model (SKFM) 10 which is a combination of a state-space model and a Hidden Markov model. Both states xₜ 12 and switching labels, Sₜ, 14 are assumed to be Markov over time, and conditioned on the states and labels. The observation likelihood is a linear Gaussian model. In contrast to the standard Kalman filter, the switching Kalman filter (SKF) introduces a discrete “switching” variable Sₜ. The intuition is that the observations may be generated by different models represented by this switching variable.

Specifically, the joint probability distribution over states ({Xₜ}), observations ({Yₜ}) and switching variables ({Sₜ}) are modeled:

$$p ( \{ {x}_{t} , {y}_{t} , {S}_{t} \} ) = [ p ( {S}_{1} ) {\prod }_{t = 2}^{T} p ( {S}_{t} \vert {S}_{t - 1} ) ] [ p ( {x}_{1} ) {\prod }_{t = 2}^{T} p ( {x}_{t} \vert {x}_{t - 1} ) ] [ {\prod }_{t = 1}^{T} p ( {y}_{t} \vert {x}_{t} , {S}_{t} ) ] ,$$

where t=1,2, . . . ,T represent discrete time binds. Conditioned on the hidden switching variable, the probability of observing the firing rate vector is given by

$$p ( {y}_{t} \vert {x}_{t} ) = {\sum }_{j = 1}^{N} p ( {S}_{t} = j ) p ( {y}_{t} \vert {x}_{t} , {S}_{t} = j ) .$$

This simply states that the probability of observing the firing rates yₜ conditioned on the kinematics xₜ is represented by a mixture model. The term p(Sₜ=j) represents the probability of being in model j at time t for each of the j=1 . . . N possible models.

The term p(yₜ|xₜ,Sₜ=j) represents the likelihood of observing the firing rates conditioned on both the kinematics and the particular model j. It is assumed that each model in the mixture is linear and Gaussian. Consequently:
p(yₜ|xₜ,Sₜ=j)=N(H_{jXt},Qⱼ),
where N(Hⱼxₜ, Qⱼ) denotes a Gaussian distribution with mean Hⱼxₜ. The matrix HⱼεR^{K×6} linearly relates the hands state to the neural firing. The noise covariance matrix is QⱼεR^{K×K}. In the case of the standard Kalman filter, N=1 and consequently there is only a single linear model of this form.

Note that the physical relationship between the firing rate and hand kinematics means there exists a time lag between then. In previous work it has been noted that the optimal lag was approximately 140 ms in the pinball task and 150 ms in pursuit tracking task. These same lags are used here for both encoding and decoding. The likelihood term should be written p(y_{t-lag}|xₜ,Sₜ=j) but to simplify the notation, the lag time is omitted in the equations.

It is assumed that the hidden states, S₁, S₂, . . . ,S_{T} form a first order Markov chain as illustrated in FIG. 1; that is,

$$p ( {S}_{t} = j ) = {\sum }_{i = 1}^{N} p ( {S}_{t} = j \vert {S}_{t - 1} = i ) p ( {S}_{t - 1} = i ) , \\ where$$

$${c}_{ij} = p ( {S}_{t} = j \vert {S}_{t - 1} = i ) , 1 \leq i , j \leq N .$$

These state transition probabilities are represented as a transition matrix C={cᵢⱼ}.
The kinematic state is also assumed to form a Markov chain represented by the system model:
p(xₜ|xₜ₋₁)=N(Aₓ_{_{t−1}},W),
where AεR^{6×6} is the coefficient matrix linearly relating hand kinematics at time t−1 to the kinematics at time t. The noise covariance matrix is WεR^{6×6} represents the uncertainty in this prediction. Note that this system model is identical to that in the traditional Kalman filter.

Encoding: In practice, all the parameters A, W, H_{1:N}, C need to be estimated from training data, in which both hand kinematics {xₜ} and firing rates {yₜ} are known, but the switching labels {Sₜ} are hidden. Therefore, all the parameters are estimated by maximizing likelihood p({xₜ, yₜ}):

$$\begin{matrix}{}^{arg max} A , W , {H}_{1 : N 1} {Q}_{1 : N} {C}^{P ( \{ xt , yt \} )} \\ {{}^{=}}^{arg max} A , W , {H}_{1 : N 1} {Q}_{1 : N} {C}^{P ( \{ xt \} ) p ( \{ yt \} \vert \{ xt \} ) .} \\ {}^{= arg max} A , W {,}^{P ( \{ xt \} ) arg max} {H}_{1 : N 1} {Q}_{1 : N} {C}^{P ( \{ yt \} \{ xt \} ) .}\end{matrix}$$

Using the linear Gaussian property of ^{P({xt})}, gives

$$\begin{matrix}{}^{arg max} A , {W}^{P ( \{ xt \} ) =} \\ {}^{arg max} A , W , {\sum }_{t = 2}^{T} [ log ( det W ) + {( {x}_{t} - A {x}_{t - 1} )}^{T} W - 1 ( {x}_{t} - {A}_{xt - 1} ) ] .\end{matrix}$$

The above minimization has a closed form solution:

$$A = {\sum }_{t = 2}^{T} {x}_{t} {{x}_{t - 1}^{T} ( {\sum }_{t = 2}^{T} {x}_{t - 1} {x}_{t - 1}^{T} )}^{- 1} , \\ W = \frac{1}{T - 1} ( {\sum }_{t = 2}^{T} {x}_{t} {x}_{t}^{T} - A {\sum }_{t = 2}^{T} {x}_{t - 1} {x}_{t}^{T} ) .$$

The other term p({yₜ}|{xₜ})=Σ_{{St}}p^{({yt,St}|{xt})} contains hidden variables {Sₜ}. While no closed form solution exists, the Expectation-Maximization (EM) algorithm offers an effective way to estimate all the parameters. Denoting θ=(H_{1:N1}Q_{1:N}C) and p(•| . . . )=p(.|{xₜ,yₜ}; θk), θₖ₊₁ is updated as:
θₖ₊₁=ar max_{Θ}E_{p({S}_{ₜ}_{}| . . . )} log p({yₜ,Sₜ}|{xₜ};θ

The details of the maximization process are known in the art and can be found, for example, in K. P. Murphy, “Switching Kalman Filter,” Compaq Cambridge Research Laboratory, Tech. Rep. 98-100, 1998. Only the updating result is shown here:

$${c}_{ij} = {\sum }_{t = 2}^{T} p ( {S}_{t} = j , {S}_{t = 1} = i \vert ... ) / {\sum }_{t = 2}^{T} p ( {S}_{t - 1} = i \vert ... ) , \\ {H}_{j} = {[ {\sum }_{t = 1}^{T} p ( {S}_{t} = j \vert ... ) {y}_{t} {x}_{t}^{T} ] [ {\sum }_{t = 1}^{T} p ( {S}_{t} = j \vert ... ) {x}_{t} {x}_{t}^{T} ]}^{- 1} , \\ {Q}_{j} = {\sum }_{t = 1}^{T} [ p ( {S}_{t} = j \vert ... ) ( {y}_{t} {y}_{t}^{T} - {H}_{j} {x}_{t} {y}_{t}^{T} ) ] / {\sum }_{t = 1}^{T} p ( {S}_{t} = j \vert ... ) ,$$

where i,j=1, . . . , N and the conditional probabilities of Sₜ, Sₜ₋₁ can be calculated using standard Dynamic Programming techniques.

Decoding (Estimation): Given the probabilistic encoding model defined above, to the problem of decoding is now discussed. In particular the following discussion involves reconstructing hand motion from the firing rates of the cells. Let x_{1:t} denote xₜ, . . . , xₜ, and similarly for y_{1:t} and S_{1:e}. It is necessary to determine the a posteriori mean xₜ=E(xₜ|y_{1:t}) that minimizes the mean square error E((xₜ−xₜ)²|y_{1:t}). This is achieved using the efficient Switching Kalman Filter algorithm which is briefly described here.

Under the SKFM framework, the posterior distribution of the state is a mixture of Gaussians, but the number of mixture components grows exponentially with tune-, that is, assume the initial p(x₁|y₁) is a mixture of N Gaussians (one for each value of S₁), then p(xₜ|y_{1:t}) is a mixture of N^{t} Gaussians (one for each possible sequence of Si, . . . ,Sₜ). The Switching Kalman Filter algorithm approximates these N^{t} Gaussians with a mixture of N Gaussians at each time step t. The fixed number N over time is maintained by “collapsing” N Gaussians into one using moment matching, which can be shown to be the optimal approximation under the criterion of minimization of relative entropy between the Gaussians.

For neural prosthetic applications the SKF algorithm is preferable to other sampling based algorithms since, by “collapsing” the posterior at each time instant, it provides both a deterministic algorithm and a probabilistic model representing uncertainty in the estimate. The uncertainty may be important for later stages of analysis. This uncertainty estimation is illustrated in the following section on experimental data analysis.

The SKF decoding algorithm proceeds as follows. At time t−1, assume that the posterior distribution p(xₜ₋₁|y_{1:t-1}) is approximated with a mixture of N Gaussians. That is,

$$p ( {x}_{t - 1} \vert {y}_{1 : t - 1} ) = {\sum }_{i = 1}^{N} {w}_{t - 1}^{i} p ( {x}_{t - 1} \vert {y}_{1 : t - 1} , {S}_{t - 1} = i )$$

where the weight wₜ₋₁=p(Sₜ₋₁=i|y_{1:t-1}) and each component
p(xₜ₋₁|y_{1:t-1},Sₜ₋₁=i)≈N(xₜ₋₁ⁱ,Vₜ₋₁ⁱ),
in which the mean and covariance of the system state are given by xt−₁=^{E}[^{x}t−I|y_{1:t}−1, Sₜ₋₁=i] and ^{vi}ₜ₋₁=Cov[xₜ−1|y_{1:t-1}, St−1=i], i=1, . . . , N respectively.

At time t, it will be shown how the posterior p(xₜ|y_{:t}) is estimated as a Gaussian mixture and how the weight, mean and covariance of each Gaussian are obtained. At first, the posterior is expanded as a linear sum:

$$\begin{matrix}p ( {x}_{t} \vert {y}_{1 : t} ) = {\sum }_{j = 1}^{N} {w}_{t}^{j} p ( {x}_{t} \vert {y}_{1 : t} , {S}_{t} = j ) \\ = {\sum }_{j = 1}^{N} {w}_{t}^{j} {\sum }_{i = 1}^{N} {g}_{t}^{ij} p ( {x}_{t} \vert {y}_{1 : t} , {S}_{t} = j , {S}_{t - 1} = i ) \\ = {\sum }_{i , j = 1}^{N} {w}_{t}^{ij} p ( {x}_{t} \vert {y}_{1 : t} , {S}_{t} = j , {S}_{t - 1} = i )\end{matrix}$$

where the weight w^{j}ₜ=p(Sₜ=j|^{y}1:t),^{g}t=P(St−1=it YI:t, St=9), and wt=wr 9t=P(^{S}t=j, St−1=ⁱtY1a),ⁱ,^{j}=1, . . . , N.

For each i and j, P(^{x}t|y_{1:t}, Sₜ=^{j}, Sₜ−1=i) is a Gaussian density function which can be calculated using the standard Kalman filter subroutine filter (shown in the Appendix). Since the Kalman filter produces a Gaussian estimate of the state:
p(xₜ|y_{1:t},Sₜ=j,Sₜ₋₁=1)=N(xᵢ^{ij},Vₜ^{ij}),
where the mean xⁱₜ^{j}=E[xt|y_{1:t}, Sₜ=^{j}, Sₜ₋₁=i] and the covariance matrix Vⁱₜ^{j}=Cov[xₜ|y_{1:t}, Sₜ=^{j}, Sₜ₋₁=ⁱ).

Let l^{it}^{^{j}}=p(yₜ|y_{1:t}−1, Sₜ=^{j}, Sₜ₋₁=i) be the likelihood of the observing the firing rate yₜ. This can be calculated as a by-product of the Kalman filter algorithm. Using the above notation, an expressions is obtained for the following weights at time t:w^{it}^{^{j=l}}^{it}^{^{j}} cij wⁱₜ−1/Σᵢⱼl^{it}^{^{j}} cijwⁱt−1, w^{j}ₜ−1, w^{j}ₜ=Σiw^{it}^{^{j}}, and g^{it}^{^{j}}=w^{it}^{^{j}}′/w^{j}ₜ.

Following the above analysis, it can be seen that p(xt|y_{1:t})=Σ^{N}i, j=1 wⁱt^{j}N(xⁱₜ^{j}, Vⁱₜ^{j}) is a mixture of N² Guassians. To reduce the number of mixture components to N, the N Gaussians that have same label at time t to form a single Gaussian are combined. That is, for each j, p(xₜ|y_{1:t}, Sₜ=j)=Σᵢgⁱₜ^{j}N (xⁱₜ^{j}, Vⁱₜ^{j}) is a mixture with N components. This is approximated as a single Gaussian by matching the mean and covariance matrix (using the subroutine collapse in the Appendix); that is,
p(xₜ|y_{1:t},Sₜ=j)≈N(xₜ^{j},Vₜ^{j}),
where the mean x^{j}ₜ=E[xₜ|^{y}_{1;t}, ^{S}^{_{t=j), and the covariance matrix Vj}}ₜ=Cov[xₜ|y1:t, ^{S}t=j],j=1, N. Finally, the desired mixture approximation to the posterior probability of the system state conditioned on the measurements is:

$$p ( {x}_{t} \vert {y}_{1 : t} ) \approx {\sum }_{j = 1}^{N} {w}_{t}^{j} N ( {x}_{t}^{j} , {V}_{t}^{j} ) .$$

In summary, the following algorithm shows how the posterior distribution p(Xt|y_{1:t}) is approximated by a mixture of N Gaussians Σj w^{j}ₜN (x^{j}ₜ, V^{j}ₜ) at time step t: From time step t−1 to t:

$$[ {x}_{t}^{ij} , {V}_{t}^{ih} , {l}_{t}^{ij} ] = filter ( {x}_{t - 1}^{i} , {V}_{t - 1}^{i} , {y}_{t} , {H}_{j} , {Q}_{j} , A , W ) , \\ {w}_{t}^{ij} = {l}_{t}^{ij} {c}_{ij} {w}_{t - 1}^{i} / {\sum }_{ij} {l}_{t}^{ij} {c}_{ij} {w}_{t - 1}^{i} , \\ {w}_{t}^{j} = {\sum }_{i} {w}_{t}^{ij} , \\ {g}_{t}^{ij} = {w}_{t}^{ij} / {w}_{t}^{j} , \\ [ {x}_{t}^{j} , {V}_{t}^{j} ] = collapse ( {\{ {x}_{t}^{ij} , {V}_{t}^{ij} , {g}_{t}^{ij} \}}_{i} ) .$$

When a single estimate and its uncertainty is desired, these can be computed from the mixture model. The state estimate xt and its error covariance Vₜ (obtained by the same approach as in collapse) are given by:

$${\hat{x}}_{t} = {\sum }_{j} {w}_{t}^{j} {x}_{t}^{j} , \\ {\hat{V}}_{t} = {\sum }_{j} {w}_{t}^{j} ( {V}_{t}^{j} + ( {x}_{t}^{j} - {\hat{x}}_{t} ) {( {x}_{t}^{j} - {\hat{x}}_{t} )}^{T} ) .$$

Examples of the SKF Method

Three sets of experiments are summarized herein below as examples of the present inventive method. The first involves the data from the pursuit tracking task in which the electrophysiological recordings were carefully sorted off-line. From this set a second, mixed, data set is constructed that simulated errors in spike sorting. Finally, data from an on-line scenario was considered where spike detection was performed in real-time; it is expected that the quality of the spike sorting will be poorer than in the pursuit tracking task resulting in mixed units.

Pursuit Tracking Task

Evaluation of the encoding and decoding performance was per-formed using cross-validation. The 182 trials were evenly partitioned into 7 segments (i.e. each segment had 26 trials). Then for each experiment, 156 trials (6 segments) were used as training data to learn the probabilistic model, and the remaining 26 trials (1 segment) were used to test the decoding accuracy. The SKFM was trained with two mixture components (N=2). This training/testing process was performed 7 times and each trial was used in the decoding stage exactly once. Despite the large amount of training data, computing the encoding model took only a few minutes. Decoding was performed in real time.

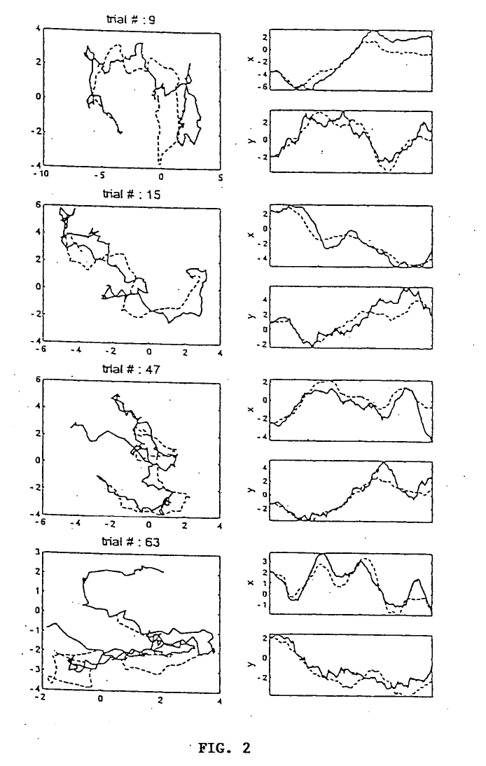

Since each testing trial was very short, the initial hand kinematics (at time 0) were let to equal the true known initial condition. The SKF was then applied to reconstruct the hand trajectory. Exemplary reconstructions for four test trials are shown in FIG. 2 in which each row represents one trial. The true hand trajectory is shown as a dashed line; the reconstruction using the SKF is shown as a solid line. The left column of FIG. 2 shows the trajectories in 2 dimensions; the right column of FIG. 2 shows the trajectories of the x and y components as a function of time.

Errors are reported in terns of mean squared reconstruction error (MSE) and the correlation coefficient (CC) for t, y position. Assume (iₜ, yₜ) is the estimate for the true position (it,yt), t=1, . . . , T then MSE and CC are defined as follows:

$$MSE = \frac{1}{T} {\sum }_{t - 1}^{T} ( {( {x}_{t} - {\hat{x}}_{t} )}^{2} + {( {y}_{t} - {\hat{y}}_{t} )}^{2} ) , \\ CC = ( \frac{{Σ}_{t} ( {x}_{t} - \overline{x} ) ( {\hat{x}}_{t} - \overline{\hat{x}} )}{\sqrt{{{Σ}_{t} ( {x}_{t} - \overline{x} )}^{2} {{Σ}_{t} ( {\hat{x}}_{t} - \overline{\hat{x}} )}^{2}}} , \frac{{Σ}_{t} ( {y}_{t} - \overline{y} ) ( {\hat{y}}_{t} - \overline{\hat{y}} )}{\sqrt{{{Σ}_{t} ( {y}_{t} - \overline{y} )}^{2} {{Σ}_{t} ( {\hat{y}}_{t} - \overline{\hat{y}} )}^{2}}} .$$

For prosthetic applications, MSE may be particularly relevant since it measures the positional accuracy of the neural reconstruction.

The waveforms for this dataset were carefully sorted off-line reducing (but not eliminating) the chance of multiple units being treated as one. The neural activity for this dataset has previously been shown to be approximately linearly related to hand kinematics. Consequently, it was expected that the SKF and Kalman filter to have similar performance.

The comparison of decoding accuracy for the SKF and Kalman Filter is shown in Table 1. The table shows the percentage of the time that the SKF was superior to (had lower reconstruction error than) the Kalman filter. The first column of results (labeled “25 (25S, 0D)”) shows that the SKF and Kalman filter had roughly the same decoding performance on this well sorted data set. The differences between the models were not statistically significant.

TABLE I
COMPARISON OF THE SKF AND KALMAN FILTER

| | # of | | | | |
|---|---|---|---|---|---|
| | units | 25(25S, 0D) | 20(15S, 5D) | 15(5S, 10D) | 13(1S, 12D) |
| MSE | superi- | 57.85% | 61.54% | 62.09% | 66.48% |
| | ority | | | | |
| | p-value | 3.35c−1 | 2.40c−3 | 1.40c−3 | 1.22c−5 |
| CC | superi- | 48.35% | 58.24% | 57.69% | 58.24% |
| | ority | | | | |
| | p-value | 7.11c−1 | 3.16c−2 | 4.54c−2 | 3.16c−2 |

Regarding notation in Table 1, for example, in the last column, “13 (1S, 12D)” denotes the use of 13 new “units” one 1 is single (1S) from the original data and 12 are double (12D). The double “units” are the result of combining pairs of single units (see text). Regarding results shown in table, for example, 66.48% denotes that the SKF has lower MSE in 66.48% of the 182 trials. 1.22e-5 is the p-value which shows the significance of the result The p-value means that if it is assumed that the Kalman filter and SKF have the same decoding accuracy, then the probability of observing lower error from the SKF 66.48% of the time is only 1.22e-5. The assumption can then be rejected. Here the p-values are calculated by the sign test.

Mixed Cells

In practical neural prosthetic applications, real-time computational constraints may mean that spiking activity is poorly sorted. To test the ability of the SKF to cope with such data, artificial spike trains were constructed by randomly selecting pairs of cells from the, pursuit tracking dataset and combining their responses. The resulting firing rate for each “double” cell was the sum of the individual firing rates. This synthetic set of cells simulated what would happen if two units were poorly separated and their waveforms were judged as coming from the same cell.

In particular, three different situations were tested with different combinations of the original 25 cells: 1) 10 units were randomly chosen from the 25, and summed their rate functions to form 5 new “units” containing spikes from pairs of original cells; 2) 20 cells were combined to form 10 doubles; and 3) 24 cells were combined to form 12 doubles. Once again, the SKFM was trained with two mixture components.

The performance of SKF was tested on this simulated experimental data and compared it with Kalman Filter. The fourth to sixth column of Table I, show that SKF outperforms the Kalman filter in terms of reconstruction accuracy and the results are statistically significant (the p-values using a sign test are all less than 5%).

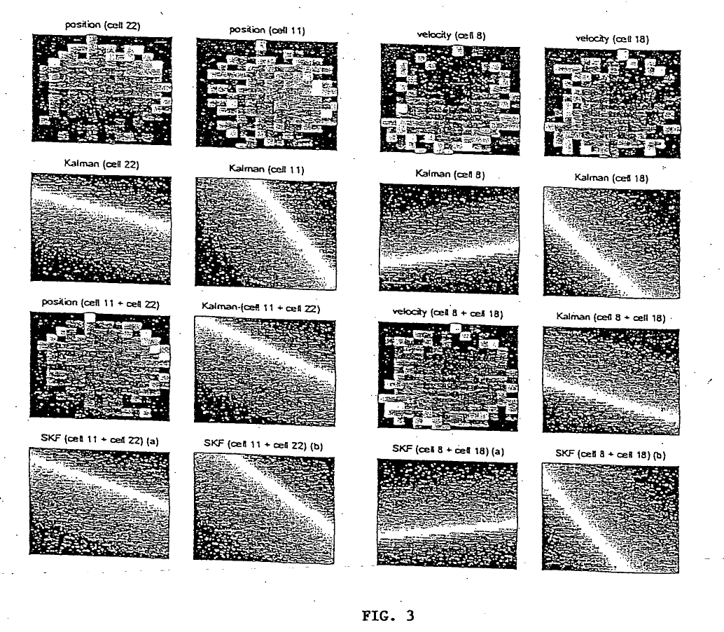

FIG. 3 provides some intuition regarding the performance of the SKFM on the double data, illustrating fitting of multiple linear models. FIG. 3 shows the empirical firing rates for a selection of cells in terms of position or velocity. Note that dark areas corresponds to no measurement while brighter areas indicate high firing rate. When the a linear Gaussian model was used to fit this data, the resulting encodings were linear (planar) as expected. FIG. 3 also shows some of the double “units” that combine the activity of two cells. In this case the-simple linear fit did not well model either of the original cells. The SKFM however fit multiple linear models (two here) and was able to approximate the original linear functions in these cases.

In the first two columns of FIG. 3, the first row shows the empirical mean firing rate distributions of two cells with respect to hand position; the second raw shows the corresponding linear fit to the data (Kalman filter model); the third row shows the empirical distribution of the sum of firing rates of these two cells and Kalman fit; and the fourth row shows the two components recovered by the SKFM when applied the combined data. It can be seen that these two linear components are very similar to the linear fits for the original two cells. The right two columns show the same behavior when firing rate is considered as a function of hand velocity. Note that the Kalman and SKF models used here fit linear models with respect to position, velocity and acceleration. The figure shows just the position or velocity component of these models.

Pinball Task

The Switching Kalman Filter model and its decoding algorithm were also tested on the data from the pinball task For this on-line task, the spike sorting was less accurate and it was hypothesized that the SKF would be more appropriate. Experimentally it was found that approximately 3.5 minutes of training data sufficed for accurate reconstruction (this is similar to the result for fixed linear filters reported in). Learning the model took approximately 1 minute on a Pentium III 866.

TABLE II
RECONSTRUCTION ACCURACY USING
THE KALMAN FILTER AND SKF

| Method | CC (x, y) | MSE (cm²) |
|---|---|---|
| Kalman | (0.82, 0., 93) | 5.87 |
| SKF | (0.84, 0.93) | 5.39 |

At the beginning of the test trial, it was assumed that no information about the hand kinematics was available and, consequently, the predicted initial condition were let to equal the average hand kinematics in training data- Both the SKF and Kalman filter were used to decode the test data and the results are reported in Table 11. The results indicated that, the SKF gave a slightly more accurate reconstruction than the Kalman Filter. Most critically, it gave an 8% reduction in mean squared error.

FIG. 4 shows the SKF reconstruction of the first 20 seconds of test data (distinct from the training data) for each component of the state variable (position, velocity and acceleration in x and y). In FIG. 4, true hand motion is shown as a dashed line and reconstruction using the SKF model is shown as a solid line. A 20 segment from a one minute test sequence is shown. It should be noted that the reconstructed trajectories are smooth and visually similar to the true trajectories (i.e. high Correlation Coefficient).

Note that the ground truth velocity and acceleration curves are computed from the position data with simple differencing. As a result these plots are quite noisy making an evaluation of the reconstruction difficult.

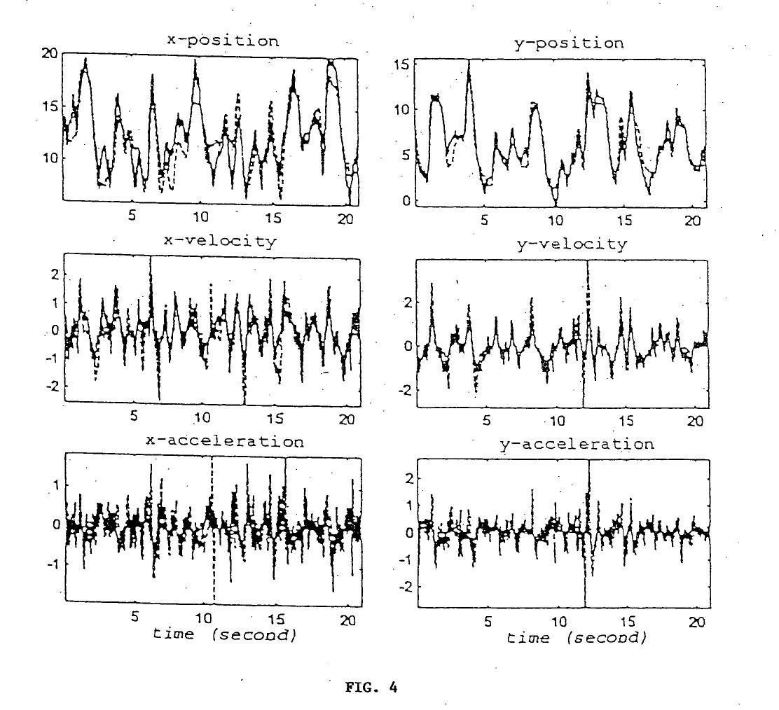

For the SKF, the posterior distribution of the state is assumed to be a mixture of Gaussians. The hand state and its uncertainty are estimated as the mean and covariance matrix of the mixture. The 95% confidence interval of this estimation is shown for both x and y-position in FIG. 5 (calculated as the (μ−2o,μ+2o), where μ,o are the mean and standard deviation in the x or y component). In FIG. 5, the first row shows the true trajectories (dashed), reconstructed trajectories (solid) and their 95% confidence range (dashdot). The second row of FIG. 5 is the normalized version obtained by subtracting the corresponding reconstruction; this shows the confidence intervals more clearly.

From FIG. 5 it can be observed that the true positions were typically within this confidence interval. Such a confidence measure may be useful for later stages of processing that may exploit the neural control signal.

Accordingly an extension of the Kalman filter is described herein for neural decoding of motor cortical activity. The approach extends the observation model of the Kalman filter as a probabilistic mixture of Linear Gaussian models. This mixture model is more general and can cope with non-Gaussian rate models and poor separation of units after spike sorting. These generalizations suggest that the approach may be more appropriate for neural control applications than previous linear methods such as the Kalman filter, linear filter, and population vector algorithm.

In particular, experiments that simulated the effect of poor spike sorting showed that the SKF performed significantly better than the traditional Kalman filter. This illustrates how the method can be applied when the assumptions of a simple linear Gaussian model are not met. To the extent that this simulation models real problems in online spike sorting, it suggests that that SKFM may be more appropriate and practical for neural prosthetic applications. Additionally, no performance decrease with well sorted data was found. Consequently, the advantages of the approach may justify the increased complexity of the method relative to the simpler Kalman filter.

The mixture model can be efficiently learned with the Expectation-Maximization algorithm using only few minutes of training data. The Switching Kalman Filter algorithm provides an efficient decoding method that gives real-time estimates of hand position in short time windows (50-70 ms here) from the firing rates of a small population of cells. Relatively accurate decoding was achieve with populations of 25-42 cells. Reconstructions of 2D hand motion were obtained for two different tasks and two different animals. The amount of data available for the Pinball task was limited thus making it impossible to state the statistical significance of the performance improvement in this case.

The hand motions explored here were more general than those in stereotyped tasks such as center-out reaching. Nevertheless, they were still simple compared with natural hand movements in daily life. For prosthetic applications, there is no reason to restrict the output of decoding to mimic natural hand motion.

Previous research has shown that the Kalman filter was more accurate at off-line decoding than linear filtering methods. The present invention using SKF is more accurate still.

It is worth noting that reasonably accurate reconstructions of hand trajectories are possible from small populations of cells (e.g. 25-42 cells as considered here). In particular, data from the Pinball experiments was used for on-line control by Semrya er al. It remains an open question how the relative performance of different decoding methods will scale to larger populations.

Accordingly, the SKFM has many of the desirable properties of the Kalman filter (e.g. linear Gaussian models (when conditioned on the switching state), full covariance model, efficient encoding and decoding), while being mole versatile and accurate. It can deal with violations (to some extent) of both the assumption of linearity and Gaussian noise. It also has an advantage over more general particle filter decoding methods in that it is computationally efficient since it does not rely Monte Carlo sampling.

Further study was performed to determine the affect of spike sorting on decoding accuracy. A single dataset was recorded and then sorted by 4 human volunteers, an automated algorithm of the inventive design, and two ‘control’ algorithms. The effect of different spike trains on decoding was evaluated by applying the Kalman filter decoding method. The Kalman filter was trained on 3 minutes of rate and kinematic data binned in 70 ms bins. Average decoding results were determined for 5 independent 1 minute data segments. The kinematics were encoded as a 6 attribute vector comprised of measured hand X/Y position, computed velocity, and computed acceleration. The present implementation differed from the implementation described hereinbefore in that it was determined that a lag of 0 ms instead of 140 ms was optimal for the studied data.

A. Recording

In a single monkey, following task training, a Bionic Technologies LLC (BTL) 100-electrode silicon array was implanted in the arm area of primary motor cortex (MI).

The recording setup for the on-line neural control of 2D cursor motion was similar to that used in M. D. Serruya, et al. “Brain-machine interface: Instant neural control of a movement signal,” Nature, vol. 416, pp. 141-142, 2002. The animal was trained to move a two-joint manipulandum on a 2D plane to control the motion of a feedback cursor on a computer screen. The simultaneous recording of hand kinematics and neural activity allows the study of motor cortical encoding of hand motion and the training of decoding methods. A recording from this animal performing a “pinball” tracking task was used in this study.

The recording used for this study was a collection of 96 independent channels where the waveform capture threshold for each channel was empirically determined at the time of recording, and was set low enough to ensure that a majority of the spiking activity was captured. The array was placed in MI, but by virtue of array design and insertion technique the location of each electrode with respect to individual neurons was unknown. Correspondingly, every channel may have had waveforms from multiple neurons. Additionally, on each channel some number of non-spike, threshold-crossing waveforms were recorded. These non-spike waveforms are referred to as “noise”. A single contiguous 600 second segment of data was extracted from a part of the recording featuring high arm movement and neural firing rates. Unlike other studies where decoding is done only on data recorded between start and stop cues, a single continuous segment was extracted where there were periods of no arm movement. Of this 600 second segment, different independent sub-segments were used for greedy model selection, to train the Kalman filter, and to test decoding accuracy.

B. Automatic Sorting

Without loss of generality, the exposition below describes sorting data on a single channel (electrode). The same process applies to all channels.

To automatically sort each channel the dimensionality of the waveforms was first reduced via principle components analysis (PCA). Expectation maximization (EM) was then used to fit a mixture of Gaussian (mixture model) to the waveform PCA coefficients. The mixture model cluster means, covariances, and membership weights were initialized by applying a spectral clustering algorithm to a subset of the data. A uniform probability noise layer was added to the EM framework and was used to identify and eliminate noise.

Let C=[\vec{ω}¹, . . . ,\vec{ω}^{N}] represent the N waveforms on a single channel, where each waveform is a vector of n voltage samples ω=[ω₁ⁱ, . . . , ωₙⁱ]^{T}ε [symbol] ⁿ In the pres 40.

The \vec{ω}ⁱ can be approximated by a linear combination of PCA bases as

$${{ω}^{->}}^{i} \approx {\sum }_{d = 1}^{D} {c}_{d} {U}_{d}$$

where D=3 bases corresponding to XX % of the variance in the data, U_{d} is the d^{th} PCA basis vector, and c_{d} are the linear coefficients. Let \vec{c}ⁱ=[c₁ⁱ, . . . , c_{D}ⁱ], then the probability P(\vec{ω}ⁱ|j) can be approximated by P(\vec{c}ⁱ|j).

Assume that all the waveforms in C were generated by exactly M neurons. Then the probability, under the Gaussian assumption, that \vec{c}ⁱ was generated by neuron j, 1<j<M is given by:

$$P ( {{c}^{->}}^{i} \vert j ) = \frac{1}{{( 2 π )}^{\frac{d}{2}} \sqrt{det ( {\sum }_{j} ) exp ( - \frac{1}{2} {( {{c}^{->}}^{i} - {μ}_{j} )}^{T} {\sum }_{j}^{- 1} ( {{c}^{->}}^{i} - {μ}_{j} ) )}}$$

Where μⱼ and

$${\sum }_{j}$$

are the mean and covariance of the PCA coefficients of the waveforms generated by neuron j.

And the probability of the channel according to the mixture model is:

$$P ( C ) = {\prod }_{i = 1}^{N} {\sum }_{j = 1}^{M} {α}_{j} P ( {{c}^{->}}^{i} \vert j )$$

where the αⱼ>0 are mixing coefficients and

$${\sum }_{j = 1}^{M} {α}_{j} = 1.$$

The EM algorithm was used to solve for optimal distribution parameters and membership weights. This involves the iterative optimization of a likelihood function like:

$$log ( L ( Θ \vert C , Y ) ) = {\sum }_{i = 1}^{N} log ( {\sum }_{j = 1}^{M} {α}_{{y}_{i}} P ( {{c}^{->}}^{i} \vert {Σ}_{j} , {μ}_{j} ) )$$

where Θ={μ₁, . . . , μ_{M}, Σ₁, . . . , Σ_{M}} is a set of unknown distribution parameters corresponding to the mean waveforms and covariance matrices for the M posited neurons,
Y={yᵢ}ᵢ₌₁^{N},1<yᵢ<M,yᵢεZ

is an array of unknown indicator variables such that yᵢ=j if \vec{c}ⁱ came from mixture component j, i.e. was generated by neuron j. These yᵢ are often called “hidden” variables.

The EM update equations are given by

$${α}_{j}^{new} = \frac{1}{N} {\sum }_{i = 1}^{N} P ( j \vert {{c}^{->}}^{i} , {Θ}^{old} )$$

$${μ}_{j}^{new} = \frac{{\sum }_{i = 1}^{N} {{c}^{->}}^{i} P ( j \vert {{c}^{->}}^{i} , {Θ}^{old} )}{{\sum }_{i = 1}^{N} P ( j \vert {{c}^{->}}^{i} , {Θ}^{old} )}$$

$${Σ}_{j}^{new} = \frac{{\sum }_{i = 1}^{N} P ( j \vert {{c}^{->}}^{i} , {Θ}^{old} ) ( {{c}^{->}}^{i} - {μ}_{j}^{new} ) {( {{c}^{->}}^{i} - {μ}_{j}^{new} )}^{T}}{{\sum }_{i = 1}^{N} P ( j \vert {{c}^{->}}^{i} , {Θ}^{old} )}$$

where Θ^{old} is the current estimate of Θ at each iteration.

The quantity P(j|\vec{c}ⁱ) is sometimes called the cluster membership weight, and is usually calculated in the following manner (here the implicit dependence on Θ is omitted):

$$P ( j \vert {{c}^{->}}^{i} ) = \frac{P ( {{c}^{->}}^{i} \vert j )}{{\sum }_{l = i}^{M} P ( {{c}^{->}}^{i} \vert l )} .$$

To account for noise a uniform outlier model with a fixed likelihood is introduced v=P(noise|\vec{c}ⁱ). This outlier models is included in the membership weight calculations in the following manner:

$$P \text{(} j \vert {\vec{c}}^{i} ) = \frac{P ( {\vec{c}}^{i} \vert j )}{{\sum }_{l = i}^{M} P ( {\vec{c}}^{i} \vert l ) + v}$$

When initializing EM there is always the issue of how to guess the initial value of Θ, given that different values of Θ can produce remarkably different clusterings due to local minima in the log likelihood function. Further, in the case of clustering spikes it was found that simple k-means or random initializations often led to solutions that were non-intuitive. To combat this Θ was initialized using spectral clustering.

The application of the algorithm to this problem is straightforward with two exceptions. The algorithm spectrally decomposes an ‘affinity matrix’, A, defined by Aᵢⱼ=exp(−∥\vec{c}ⁱ−\vec{c}^{j}∥²/2σ²) if i≠j and Aᵢᵢ=0 to find groups of waveforms that are ‘closely bunched’. The first exception is the free affinity parameter σ which was set empirically. The second exception is the fact the size of the affinity matrix grows as the square of the number of waveforms. On most channels there were too many waveforms to create an affinity matrix between all of them, so a random subset of 1000 waveforms was selected and used. The resulting cluster assignments were used to initialize the mixture means, covariances, and membership weights.

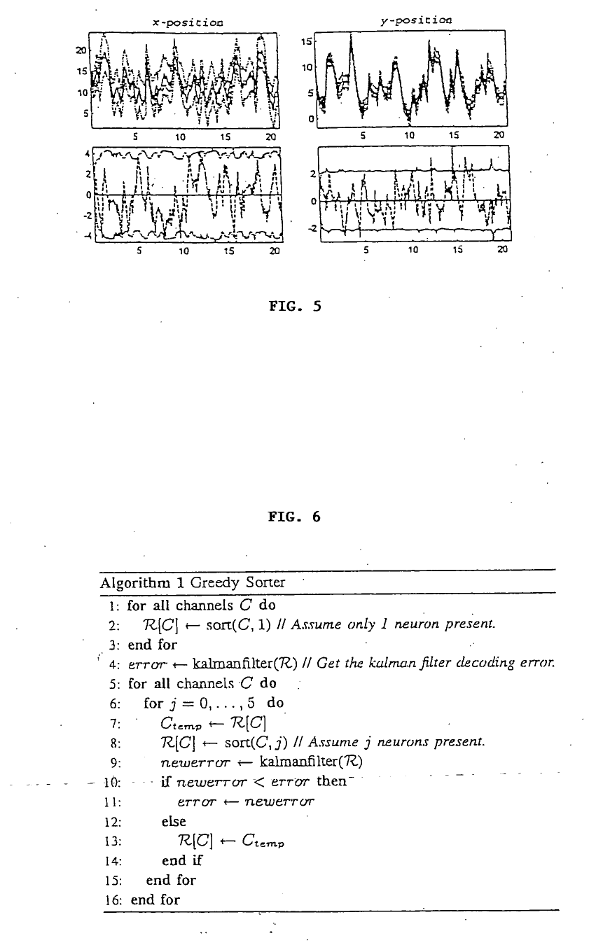

With mixture model clustering there is also the issue of model selection, taken here to mean determining how many clusters are in the data; i.e. the number of units on a channel. This corresponds to determining the number of densities, M, in the mixture model. A greedy approach was employed as shown in FIG. 6.

In the call to sort(C, j), runs the mixture model fitting procedure on channel C assuming j mixture components. The sort procedure returns a firing rate function as described below. The method sorts each channel of a recording, positing differing numbers of units, then selects the number of units that maximizes decoding performance. Note that the method can posit zero units on a channel which allows the method to exclude channels that do not contribute to accurate decoding. Note further that these excluded channels may well contain valid waveforms but the activity of the cell might be unrelated to the decoding task.

C. Human Sorting

Previous research has found that expert human sorters produced different spike trains given the same recording, however the way this affected decoding results was not studied. Volunteers were asked to sort the same dataset in order to determine how subjective variability in spike trains affects decoding results and to establish a baseline against which to judge the inventive algorithm.

Four subjects, all graduate students or postdoctoral researchers and all expert spike sorters, were given the recording and asked to sort it using any tool at their disposal. The subjects were instructed to sort in the way they thought would maximize decoding performance. All subjects used Plexon's Offline sorter software to sort the dataset. Units were identified and spikes were assigned to them by manually cluster cutting waveforms projected into a 2 dimensional PCA space. The resulting spike trains were decoded and the results are displayed as A, B, C, and D in Table 1.

D. Control Sorting

To better understand how the present method and the volunteers performed, results were compared to spike trains produced by both randomly sorting or not sorting at all. No sorting (‘None’ in Table I) means that all waveforms on each channel were attributed to a single neuron. Randomly sorting (‘Random’ in Table I) means that on each channel three neurons were posited and waveforms were randomly attributed to them with equal probability.

E. Rate Estimation

A common approach to neural decoding exploits a relationship between the firing rates of individual neurons and some stimulus or motor variable (hand position in the present case). It is common to ‘bin’ spikes within fixed time windows to produce an estimate of firing rate for use in decoding. For instance, the Kalman filter as applied in earlier studies posits and exploits a linear Gaussian relationship between hand kinematics and firing rate.

Decoding performance was examined given two different estimates of the firing rate. One is the usual maximum likelihood clustering interpretation, the results for which are given as ‘Auto Max’ in results Table I. The second, ‘Auto Weighted’ in the same table, attempts to account for some of the spike train ambiguities in a probabilistically sound way.

Let

$$B = {\sum }_{i = 1}^{C} {M}_{i}$$

be the number of cells identified on all channels where Mᵢ; is optimal the number of units identified for each channel. Let zₖ=[zₖ¹, zₖ², . . . , zₖ^{B}] be the firing rate of all B cells identified in the recording in time bin k where zₖ^{j} is the firing rate of cell j. Let \vec{c}ⁱ,0<i<K be the PCA representation of the K waveforms recorded between the start and end times of bin k.

Then, under the mixture model, the maximally likely firing rate, ‘Auto Max’, is computed as usual:

$${z}_{k}^{j} = {\sum }_{i = 0}^{K} \{ \begin{matrix}1 & if & j = arg {max}_{m} P ( m \vert {\vec{c}}^{i} ) ; \\ 0 & otherwise & \end{matrix}$$

A motivation for adapting a probabilistic, mixture model sorting algorithm was to leverage the characterization of uncertainty in the decoding algorithm. The ‘Auto Weighted’ decoding used the same Kalman decoding framework but the firing rate was computed as:

$${z}_{k}^{j} = {\sum }_{i = 0}^{K} P ( j \vert {\vec{c}}^{i} )$$

In both estimates of firing rate P(j|\vec{c}ⁱ)=0 if \vec{c}ⁱ was not recorded on the same channel where neuron j was identified.

This weighting scheme accounts for spikes that are difficult to attribute to any single neuron. It does so by contributing a ‘partial spike’ or ‘weight’ to the firing rates of every neuron that could have generated them. In the clustering context, this is equivalent to eschewing a maximum likelihood clustering criteria, and instead finding a way to cope with the fact that a point may be inherently ambiguous and should be treated is if it belongs to several clusters simultaneously. In this ‘auto weighted’ rate estimation, the mixture likelihood was used given a spike P(j|\vec{c}ⁱ) as the ‘partial spike’ weight contribution to neuron j.

Results of Automatic Sorting

Table III summarizes the decoding results and lists average Kalman filter decoding results for five independent one minute spike trains as sorted by four human subjects, two control algorithms (‘none’ and ‘random’), and the illustrative algorithm according to the present invention with two different firing rate estimates (‘max’ and ‘weighted’). In the table, ‘None’ posits one noiseless unit per channel. ‘Random’ posits three units per channel and assigns waveforms to each unit uniformly at random. ‘Ave. Human’ is the average of subjects ‘A’, ‘B’, ‘C’, and ‘D’. ‘Auto Max’ is the result for the algorithm described in this paper with the normal, maximum likelihood estimate of firing rate. ‘Auto Weighted’ uses the same clustering as ‘Auto Max’ except that a probabilistically ‘weighted’ estimate of firing rate is used instead. ‘None’ and ‘Random’ are shown for comparison. Reported are the total number of neurons and spikes identified, the correlation coefficients between the decoded and the true x and y hand position, and the mean square error (MSE) between the same. It was found that the present automatic sorter did as well or better than the best human sorter.

TABLE III

| Subject | Neurons | Spikes | Corr-Coeff (x, y) | MSE (cm₂) |
|---|---|---|---|---|
| A | 107 | 757674 | (0.91, 0.90) | 11.45 ± 1.39 |
| B | 96 | 335656 | (0.84, 0.87) | 16.16 ± 2.38 |
| C | 78 | 456221 | (0.89, 0.89) | 13.37 ± 1.52 |
| D | 88 | 642422 | (0.90, 0.89) | 12.37 ± 1.22 |
| Ave. Human | 92 | 547993 | (0.89, 0.89) | 13.46 ± 2.54 |
| Random | 288 | 860261 | (0.89, 0.87) | 13.28 ± 1.54 |
| None | 96 | 860261 | (0.91, 0.89) | 12.78 ± 1.89 |
| Auto Max | 114 | 625861 | (0.92, 0.90) | 11.31 ± 1.33 |
| Auto Weighted | 114 | 625861 | (0.92, 0.89) | 11.30 ± 1.15 |

It should be noted that the average human spike train is worse for decoding than randomly sorting and that, on average, the inventive automated algorithm produces the best spike trains for decoding among the listed options.

The fact that human spike sorters produced spike trains that are worse for decoding than no sorting at all is an unexpected result. With the exception subject ‘A’, the subjects generally erred toward missing valid spikes in lieu of attributing non-spikes to neurons. This sorting strategy could result in problematically low firing rates for use in decoding. Subject ‘A’ identified more spikes than did the automated algorithm, but decoding results for his spike trains were slightly worse than for those from the automated algorithm. This could be due to the fact that the automated algorithm found more units and presumably those units had better tuning properties.

This was not always the case. While the automated algorithm improved decoding accuracy, the structure of the relationship between firing rate and kinematic variables for the automatically sorted data did not always appear better upon visual inspection. The Kalman filter assumes a linear relationship between firing rate and kinematics, thus units whose tuning properties were less noisy and perhaps more cleanly linear were expected. Only in some cases were such units seen.

For decoding it might be more important to find units that are well correlated to the kinematic variables than to find precisely the unique units being recorded. This differs substantially from the objectives of spike sorting for neuroscience at large, and resulted in some unusual but effective clusterings. Consider a channel containing clearly separable clusters of waveforms from two different neurons. If the combination of the tuning properties of the two units are ‘more linear’ than either of the two units individually, then the automated algorithm tended to cluster them together into one, linearly tuned unit.

Additionally, it has been observed that ‘discarding’ certain units sometimes improves decoding results. The present inventive algorithm discarded some units, and indeed some channels wholly, by labeling all activity on poorly correlated channels to be noise. The sequential greedy nature of this algorithm may remove more units from consideration than is necessary. It is believed that the observed improvement in decoding results may have come from the elimination of superfluous data in this way.

In another illustrative embodiment of the present invention, on-line, closed-loop, neural control of cursor motion using the Kalman filter is described. In this embodiment, a monkey moves a cursor on a computer monitor using either a manipulandum or their neural activity recorded with a chronically implanted micro-electrode array. The applicability of the Kalman filter for neural prosthesis applications is suggested, however it was observed that the decoded cursor position was nosier under brain control as compared with manual control using the manipulandum. To smooth the cursor motion without decreasing accuracy, a method is proposed that smoothes the neural firing rates. This smoothing method is described below and its validity is quantitatively evaluated with recorded data.

In the present embodiment, the Kalman filter is demonstrated in a closed-loop neural control task. The experimental paradigm of the illustrative embodiment requires a monkey to control the two-dimensional (2D) motion-of a feedback cursor viewed on a video monitor. The animal's task was to move the cursor to hit targets that appeared at random locations on the screen. In the experiment, a single macaque monkey was implanted with an electrode array and the Kalman filter was used to decode the neural activity in real time to estimate the cursor position. By closed-loop, it is meant that the brain directly controls the cursor and the visual feedback to the animal about the cursor position closes the loop.

To evaluate the performance of the closed-loop method the animal's ability to hit targets with both the Kalman filter was compared with a simple linear regression method which has been widely used for neural decoding. The accuracy of the method in off-line data analysis was also evaluated. While advantages of the Kalman filter have been described hereinbefore, here it is shown that the Kalman filter outperforms the linear regression method in the closed-loop neural control task.

Though accurate and efficient, the estimates produced by the Kalman filter are not as smooth as those observed under manual control using a manipulandum. While the significance of this is unclear from the viewpoint of neural control, various methods to produce smooth neural decodings that more closely approximate the motion under manual control are explored. In previous work the decoded hand position produced by the linear regression method was smoothed using a simple windowed average method [2]. In the present embodiment, an on-line smoothing approach is proposed that instead smoothes the neural firing rate data. This results in smooth reconstructions of hand motion while providing a good balance between bias (accuracy) and the variance (smoothness).

A. Experimental Paradigm

Simultaneous recordings of spikes are acquired from an array consisting of 100 micro-electrodes chronically implanted in the arm area of primary motor cortex (MI) in a Macaque monkey. Multi-unit firing activity on each channel is detected using simple filtering and thresholding. The behavioral paradigm described in M. D. Serruya, et al., “Brain-Machine Interface: Instant neural control of a movement signal,” Nature, vol. 416, pp. 141-142, 2002 is used for two tasks described below.

Manual-control task: The behavioral task for the monkey is referred to as a step-tracking task, or more intuitively, a pinball task which is designed to test direct neural control performance. During the task, a target dot was shown on the screen in front of the monkey and the monkey moved a manipulandum on a 2D tablet that was parallel to the floor. The position of the manipulandum (same as the hand) was shown as a feedback cursor on the same screen. The monkey was required to move this cursor to “hit” the target (within a pre-specified distance). When the target was acquired, it disappeared and then reappeared in a new random location. Each time the target appeared, the monkey moved to hit the new location.

The hand trajectory and the neural activity were recorded simultaneously. The spiking activity was detected via empirically determined threshold settings and a firing rate was computed using non-overlapping 70 ms time bins [6]. The position, velocity, and acceleration of the hand were also computed every 70 ms.

Neural-control task: In the closed-loop neural control task the experiment remained the same except that the motion of the cursor was controlled by the decoded neural signals. This decoding was performed using a Kalman filter. To quantitatively compare with related work, the same procedure was repeated using a linear regression method for decoding. These two methods are described below. Recordings were made during experiments over several months and details of the experiments are described in Section III.

B. Statistical Methods

1) Kalman filter: Here, the Kalman filter model and its decoding algorithm are briefly described. In general, decoding involves estimating the state of the hand at the current instant in time; i.e. xₖ=[x, y, vₓ, v_{y}, aₓ, a_{y}]^{T}ₖ representing x-position, y-position, x-velocity, y-velocity, x-acceleration, and y-acceleration at time tₖ=kΔt where Δt=70 ms in the experiments. The Kalman filter model assumes the state is linearly related to the observations zₖεŹ^{C} which here represents a C×1 vector containing the firing rates at time tₖ for C×1 observed neurons; the state itself is linearly related over time as well.

Such assumptions can be described in the following two equations:
Firing rate zₖ=Hxₖ+q_{k′} (1)
State xₖ=Axₖ₋₁+w_{k′} (2)
where k=1,2, . . . , M, M is the number of time steps in the trial, and Hε [symbol] ^{Cx6}, Aₖε [symbol] ^{6×6 linear coefficient matrices. The noise terms q}ₖ, wₖ are assumed zero mean and normally distributed, i.e. qₖ, ˜N(0, Q), Qε [symbol] ^{CxC}, wₖ˜N(0, W), Wε [symbol] ^{6×6}. These equation linear Gaussian model from which the state and its uncertainty can be estimated recursively using the Kalman filter algorithm.

2) Linear regression method: The linear regression method (also referred to as a discrete Wiener filter) has been used in the decoding of neural signals in motor cortex, and particularly in closed-loop neural control tasks. It is briefly described here as the baseline method for comparison.

The linear regression method reconstructs hand position as a linear combination of the firing rates over some fixed time period; that is, where xₖ is the x-position (or, equivalently, the y-position) at time tₖ=kΔt (Δt=70 ms), k=1, . . . , M M, where M is the number of time steps in a trial, a is a constant offset, z iₖ₋ⱼ is the firing rate of neuron i at time tₖ₋ⱼ, and fij are the filter coefficients. The coefficients can be learned from training data using a simple least squares technique. In the present experiments, N=10 which means that the hand position is determined from firing data over 0.7 s.

Results of Closed-Loop Control

A. Off-Line Reconstruction

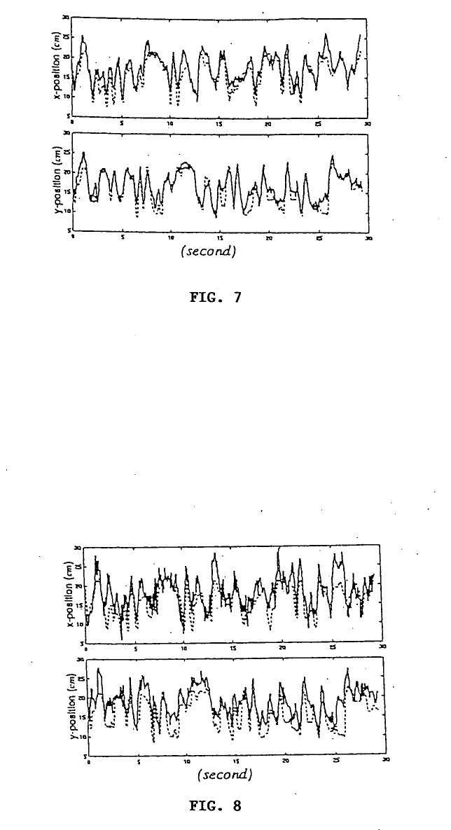

For off-line analysis six experiments were performed. For each experiment the recorded data was divided into separate training and testing datasets. Each dataset (training or testing) was approximately 1 to 2 minutes long. For each experiment both the Kalman filter and linear regression models were trained using the training set, then reconstructed trajectories were computed for the corresponding test dataset. Some example reconstructions (for the 5th test dataset) are shown in FIGS. 7 and 8. As a summary, Table IV shows the decoding results in all six test datasets where the correlation, CC, and the mean squared error, MSE, are used to quantitatively describe the accuracy. Note that the number of recorded cells differed from day to day and consequently testing was performed using the model trained on data from that same day. The significant increase in the number of cells for the last two experiments was due to the implantation of a new array.

TABLE IV
Off-Line Reconstruction

| | Kalman filter | | linear regression | |
|---|---|---|---|---|
| # of cells | CC | MSE(cm²) | CC | MSE(cm²) |
| 23 | (0.79, 0.82) | 13.0 | (0.70, 0.72) | 18.9 |
| 30 | (0.88, 0.79) | 10.6 | (0.85, 0.72) | 12.2 |
| 36 | (0.75, 0.74) | 19.0 | (0.77, 0.64) | 19.2 |
| 26 | (0.71, 0.76) | 20.1 | (0.71, 0.74) | 19.3 |
| 69 | (0.88, 0.89) | 9.7 | (0.72, 0.78) | 28.1 |
| 69 | (0.86, 0.88) | 10.6 | (0.71, 0.80) | 15.9 |

Table IV, shows that the Kalman filter has better decoding performance than the linear regression method for both criteria. This is consistent with previous observations on different datasets. Note that the linear regression results are reported without the post hoc smoothing described below; the accuracy is consistently lower with smoothing.

B. Closed-Loop Neural Control

Each closed-loop experiment had two phases. The training phase was the same as in the off-line experiments. The subject's hand movement and neural firing were recorded and used to train the two models.

After building the model, the second phase was performed which involved closed-loop neural control. In this stage, the motion of the feedback cursor was controlled by either the Kalman filter or the linear regression method. Four experiments were performed using the Kalman filter and three using the linear regression method. The results are summarized in Table V.

TABLE V
Closed-Loop Neural Control

| | Kalman filter | | | linear regression | | |
|---|---|---|---|---|---|---|
| # of cells | time | targets | rate | time | targets | rate |
| 17 | 60 sec | 38 | 38/sec | | | |
| 30 | 105 sec | 55 | 31/sec | 58 sec | 24 | 25/min |
| 36 | 57 sec | 28 | 29/sec | 42 sec | 15 | 21/min |
| 69 | 45 sec | 28 | 37/sec | 60 sec | 22 | 22/min |

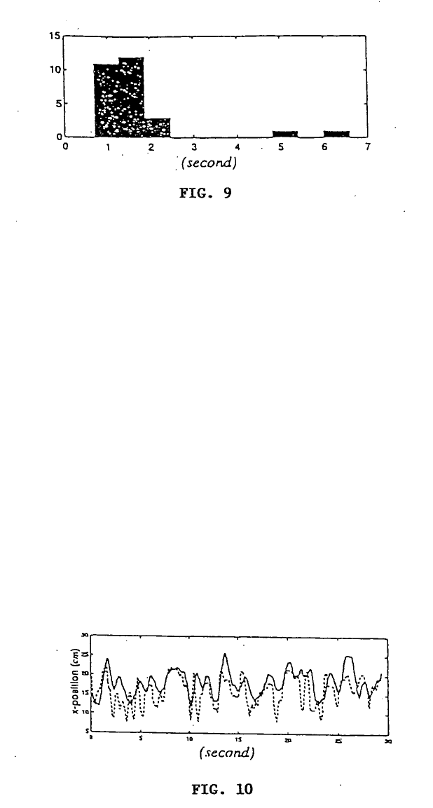

Table V shows the total time for the experiment, the number of targets hit during this time, and the rate at which the animal hit the targets. For more detail, a histogram of the time required to hit the targets using the Kalman filter is shown for the fourth experiment in FIG. 9. It can be seen with reference to FIG. 9 that most of time the target is promptly acquired, while there are a few outliers for which it took the animal 5-6 s to acquire the target. To provide a criterion for the comparison of the Kalman filter and the linear regression method the number of targets hit per unit time are considered (see the columns under “rate” in Table V). For these initial experiments it is observed that the monkey appears to consistently acquire targets at a higher rate with the Kalman filter as compared with to the linear regression method.

The results suggest that the Kalman filter decoding of motor cortical activity is appropriate for neural prosthesis applications and preliminary results suggest that it is more accurate in this context than traditional linear regression methods.

It is noted that the linear regression reconstruction is very erratic and requires post hoc smoothing to produce useful closed-loop cursor control. This smoothing was performed using a moving average filter where the average is computed over 10 time bins (700 ms). FIG. 10 is an example of the reconstruction of the x hand position after smoothing. In FIG. 10, dashed lines represent true trajectory and solid lines represent reconstructed trajectory. The figure illustrates that the averaging procedure introduces a time lag that contributes to a decrease in decoding accuracy in off-line experiments. Quantitatively the decoding accuracy was obtained: CC=(0.55, 0.58), MSE=33.4. For comparison, the accuracy of the linear regression method without smoothing for this data was CC=(0.72, 0.78) and MSE=28.1.

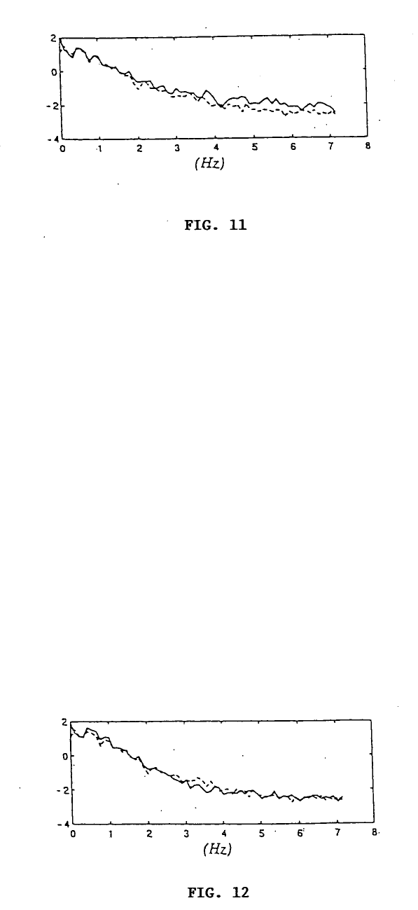

The state equation in the Kalman filter links the estimate at neighboring time steps and this helps to smooth decoding results. While the Kalman filter was less jerky than the linear regression method without smoothing, the decoded cursor motion was still less smooth than the cursor motion observed during manual control. This can be seen in FIG. 11 where the power spectra of the true and reconstructed trajectories are shown overlaid. FIG. 11 shows the logarithm of power spectra of true x-position and reconstructed x-position in the 5th test dataset using the Kalman filter. Here again, dashed line represents true position and solid line represents the reconstructed position. It can be seen that the reconstructed trajectory has larger power in the high frequencies (above 4 Hz) than does the true motion.

It is suspected that the jerkiness in the reconstruction is due to the finite approximation of the neural firing rate derived from the 70 ms time bins. With more cells, such jerkiness is less likely to present a problem. However, in the near term the population size for neural prosthesis applications is unlikely to be significantly larger than that used here.

To produce a smoother control signal, a weighted low-pass filter of the firing rate signals is proposed. In order to minimize the time lag introduced by smoothing the firing rate, current measurements are weighted more heavily. Assume for the i^{th} cell, the observed firing rate at the k^{th} time step is: zik; then the smoothed firing rate at the same time step is:
w₁x_{k-N+1}ⁱ+w₂z_{k-N+2}ⁱ+ . . . +w_{N-1}zₖ₋₁ⁱ+w_{N}zₖⁱ,
where N is the length of the smoothing window, {w₁, . . . , w_{N}} are the weights of firing rates. Note for neural control the smoothing window cannot look forward in time without introducing a time lag.

FIG. 12 shows the power spectra for the true and reconstructed trajectories where the reconstruction uses the smoothed firing rates in both training and test datasets. FIG. 12 presents the logarithm of power spectra of true x-position and reconstructed x-position in the 5th test dataset using the Kalman filter with smoothed firing rates. The dashed line represents true position and the solid line represents reconstructed position. Here N=5 and the linear weights are wᵢi=1, . . . 5. The reconstructed trajectory has similar power in the high frequencies to the true one suggesting that smoothing the firing rate also smoothes the estimated reconstruction. In contrast to the smoothed linear regression results, such a large a decrease in accuracy is not observed. Quantitatively, the decoding accuracy of CC=(0.84, 0.85), MSE=12.0 for the smoothed case was obtained. For the unsmoothed firing rates the accuracy was CC=(0.88, 0.89) and MSE=9.7.

Accordingly, through off-line and on-line experiments it was demonstrated that the Kalman filter decoding method can be successfully exploited for closed-loop 2D neural motor control tasks. Furthermore, the Kalman filter was compared with a linear regression method with respect to closed-loop performance. For this limited dataset the results showed that the Kalman filter was superior (in terms of number of targets hit in unit time). An on-line approach for smoothing the firing rate has also been shown to produced smoother off-line hand reconstructions without significant loss of decoding accuracy.

Although embodiments of the present invention are described herein in terms of decoding neural signals for hand kinematics, persons having ordinary skill in the art should understand that the decoding and control methods of the present invention can be applied to decoding neural signals for virtually any motor function without departing from the scope of the present invention. It is envisioned that the methods of decoding and control of the present invention could also be applied, for example, to non-motor neural functions such as cognitive and emotional brain signal decoding within the scope of the present invention.

Although the various illustrative embodiments of the invention are described herein in terms of neural signals, i.e. spikes acquired on one of a plurality of channels of implanted electrodes, persons having ordinary skill in the art should appreciate that a variety of other spike acquisition methods can be used without departing from the spirit and scope of the present invention. For example, it is envisioned that remote detection methods or any method of collecting spikes representing neural firing can be used in various embodiments of the present invention.

Although the various illustrative embodiments of the present invention are described herein in terms of neuro-prosthetic applications, persons having ordinary skill in the art should understand that embodiments of the present invention can be implemented in a vast number of alternative applications. For example, it is envisioned that neural signal decoding and control methods according to the present invention can be used to control computer applications, external mechanical devices, or virtually any remotely controllable device.

Although the invention has been shown and described herein with respect to exemplary embodiments thereof, various other changes, omissions and additions in the form and detail thereof may be made therein without departing form the spirit and scope of the invention.

What is claimed is:

1. A method of decoding neural signals comprising the steps of:
modeling neural firing rates as a Gaussian mixture having Gaussian components wherein the mean of each component is a linear function of motor kinematics; and
modeling the probability of each component.

2. A method of decoding neural signals comprising the steps of:
modeling a motor activity as a kinematic state vector;
modeling neural firing rates detected during said motor activity as an observation vector;
implementing a switching Kalman filter to model the probabilistic relationship between said state vector and said observation vector.

3. A neural signal decoding system comprising:
a plurality of electrodes adapted for sensing neural signals in the motor cortex of an animal brain;
an amplifier in electrical communication with said electrodes and adapted to amplify said neural signals;
a recorder in electrical communication with said amplifier and adapted to sample and record amplified neural signals therefrom, said recorder storing a time stamp and electrode identifier for each sample;
a computer system in communication with said recorder and programmed to model neural firing rates as a Gaussian mixture having Gaussian components wherein the mean of each component is a linear function of motor kinematics, and modeling the probability of each component.

4. A neural signal decoding system comprising:
a plurality of electrodes adapted for sensing neural signals in the motor cortex of an animal brain;
an amplifier in electrical communication with said electrodes and adapted to amplify said neural signals;
a recorder in electrical communication with said amplifier and adapted to sample and record amplified neural signals therefrom, said recorder storing a time stamp and electrode identifier for each sample;
a computer system in communication with said recorder and programmed to:
model motor activity as a kinematic state vector;
model neural firing rates detected during said motor activity as an observation vector; and
implement a switching Kalman filter to model the probabilistic relationship between said vector and said observation vector.

5. The neural signal decoding system according to claim 3 further comprising means for detecting and recording time-stamped kinematic data corresponding to said animal's motor activity while said neural signals are detected by said electrodes.

6. A method of automatically separating neural signal wave-forms from multiple cells comprising the steps of:
acquiring a set of neural signal waveforms from a detector channel;
reducing dimensions of said waveforms using Principal Component Analysis (PCA) to generate PCA coefficients of said waveforms;
fitting a Gaussian mixture model to said PCA coefficients using an Expectation Maximization Process;
wherein each mixture model cluster mean, covariance and membership weight are initialized by applying a spectral clustering algorithm to a portion of said waveform.

7. The method according to claim 6 further comprising the step of:
identifying noise by adding a uniform probability noise layer in said expectation maximization process.

8. The method according to claim 6 further comprising the step of:
determining how many densities are in said mixture model using a greedy sorter.

9. The method according to claim 8 wherein using said greedy sorter comprises the steps of:
sorting each channel of a recording positing different numbers of units;
determining the Kalman filter decoding error for each number of units for each channel; and
selecting the number of units that maximizes decoding performance for each channel.

10. A system for automatically decoding neural signal waveforms comprising:
means for acquiring a set of neural signal waveforms from a detector channel; and
computer means in communication with said means for acquiring, said computer means being programmed to reduce dimensions of said waveforms using Principal Component Analysis (PCA) to generate PCA coefficients of said waveforms and fit a Gaussian mixture model to said PCA coefficients using an Expectation Maximization Process;
wherein each mixture model cluster mean, covariance and membership weight are initialized by applying a spectral clustering algorithm to a portion of said waveform.

11. A method of controlling an external event with interpreted neural signals comprising the steps of:
detecting said neural signals in a subject's brain;
automatically decoding said neural signals using a Switching Kalman Filter;
altering the external event according to said decoded neural signals;
providing feedback of said altered external event to said subject.

12. The method according to claim 11 further comprising the step of:
smoothing firing rates of detected neural signals using a weighted low pass filter.

13. A system for controlling an external event with interpreted neural signals comprising:
sensor means for detecting said neural signals in a subject's brain; and
computer means in communication with said sensor means, said computer means being programmed for automatically decoding said neural signals using a Switching Kalman Filter.
